(19) United States
(12) Patent Application Publication
(10) Pub. No.: US 2024/0423648 A1
(43) Pub. Date: Dec. 26, 2024

(54) SURGICAL CUTTING BLOCK INCLUDING MULTIPLE CUT GUIDES

(71) Applicant: Fusion Orthopedics USA, LLC, Mesa, AZ (US)

(72) Inventors: Adam J. Cundiff, Gilbert, AZ (US); Nathan G. Peterson, Gilbert, AZ (US); Kolby K. Black, Spanish Fork, UT (US); Mark William Roberts, JR., Gilbert, AZ (US); Eli W. Jacobson, Chandler, AZ (US)

(73) Assignee: Fusion Orthopedics USA, LLC, Mesa, AZ (US)

(21) Appl. No.: 18/807,737

(22) Filed: Aug. 16, 2024

Related U.S. Application Data
(62) Division of application No. 17/182,125, filed on Feb. 22, 2021, now Pat. No. 12,064,127.
(60) Provisional application No. 63/115,187, filed on Nov. 18, 2020.

(51) Int. Cl. A61B 17/17; A61B 17/15; A61B 17/56; A61B 17/84

(52) U.S. Cl. CPC A61B 17/17; A61B 17/151; A61B 17/152; A61B 17/1775; A61B 17/56; A61B 2017/564; A61B 2017/565; A61B 2017/567; A61B 2017/568; A61B 17/842

(57) ABSTRACT

Various surgical cutting blocks are presented herein. One surgical cutting block includes a cutting platform including a positioning aperture formed on the cutting platform in which the positioning platform divides the cutting platform into first and second sides and the first side and/or the second side includes multiple single cut guides formed thereon. Another surgical cutting block includes a cutting platform including a positioning aperture formed on the cutting platform in which the positioning platform divides the cutting platform into first and second sides and the first side and/or the second side incudes a double cut guide formed thereon. Yet another surgical cutting block includes a cutting platform including a positioning aperture formed on the cutting platform in which the positioning platform divides the cutting platform into first and second sides and the first side and/or the second side includes a multi-cut guide formed thereon.

### REFERENCE TO RELATED APPLICATION

This application is a Divisional of and claims priority to U.S. patent application Ser. No. 17/182,125, now U.S. Pat. No. ______, filed on Feb. 22, 2021, which claims priority to and the benefit of U.S. Provisional Patent Application No. 63/115,187, filed on Nov. 18, 2020. The entire disclose of each of the foregoing applications are hereby incorporated herein by reference in their entirety.

### FIELD OF THE TECHNOLOGY

The present technology relates generally to surgical instruments, and more particularly to, a surgical cutting block including a plurality of cut guides.

### BACKGROUND

Different surgical procedures utilize different instruments and techniques. In an osteotomy for correcting a bunion, for example, the surgeon makes multiple cuts to the bone to realign the joint using multiple surgical cutting blocks that each include a single cutting guide, That is, each time that a new cut is needed to be made to the bone, the surgeon must move the surgical cutting block to a new location or use a different surgical cutting block at the new location to make the cut, both of which are time consuming and/or an inefficient use of surgical instrumentation.

### SUMMARY

Various embodiments provide a surgical cutting block including a plurality of cut guides. One surgical cutting block includes a cutting platform including a positioning aperture formed on the cutting platform in which the positioning platform divides the cutting platform into a first side and a second side. The surgical cutting block further includes plurality of single cut guides formed on the first side and/or the second side.

Another surgical cutting block includes a cutting platform including a positioning aperture formed on the cutting platform in which the positioning platform divides the cutting platform into a first side and a second side. The surgical cutting block further incudes a double cut guide formed on the first side and/or the second side.

Yet another surgical cutting block includes a cutting platform including a positioning aperture formed on the cutting platform in which the positioning platform divides the cutting platform into a first side and a second side. The surgical cutting block further includes a multi-cut guide formed on the first side and/or the second side.

### BRIEF DESCRIPTION OF THE DRAWINGS

To readily understand the advantages and benefits of the technology, a more particular description of the technology briefly described above will be rendered by reference to specific embodiments that are illustrated in the appended drawings. Understanding that these drawings depict typical embodiments of the technology, and are therefore not to be considered to be limiting of its scope, the technology will be described and explained with additional specificity and detail through the use of the accompanying drawings, in which:

FIG. 1 is a diagram illustrating one embodiment of a surgical cutting block including a plurality of single cut guides;

FIG. 2 is a diagram illustrating another embodiment of a surgical cutting block including a plurality of single cut guides;

FIG. 3 is a diagram illustrating yet another embodiment of a surgical cutting block including a plurality of single cut guides;

FIG. 4 is a diagram illustrating still another embodiment of a surgical cutting block including a plurality of single cut guides;

FIG. 5 is a diagram illustrating one embodiment of a surgical cutting block including a plurality of double cut guides;

FIG. 6 is a diagram illustrating another embodiment of a surgical cutting block including a plurality of double cut guides;

FIG. 7 is a diagram illustrating yet another embodiment of a surgical cutting block including a plurality of double cut guides;

FIG. 8 is a diagram illustrating still another embodiment of a surgical cutting block including a plurality of double cut guides;

FIG. 9A is diagram illustrating a top view of one embodiment of a surgical cutting block including a single cut guide and a double cut guide;

FIG. 9B is diagram illustrating a side profile of the surgical cutting block illustrated in FIG. 9A;

FIG. 9C is a diagram illustrating side view of the surgical cutting block illustrated in FIGS. 9A and 9B;

FIG. 10 is a diagram illustrating another embodiment of a surgical cutting block including a single cut guide and a double cut guide;

FIG. 11 is a diagram illustrating one embodiment of a surgical cutting block including a plurality of single cut guides and a double cut guide;

FIG. 12 is a diagram illustrating another embodiment of a surgical cutting block including a plurality of single cut guides and a double cut guide;

FIG. 13 is a diagram illustrating one embodiment of a surgical cutting block including a plurality of double cut guides and a single cut guide;

FIG. 14 is a diagram illustrating another embodiment of a surgical cutting block including a plurality of double cut guides and a single cut guide;

FIG. 15 is a diagram illustrating one embodiment of a surgical cutting block including a plurality of double cut guides and a plurality of single cut guides;

FIG. 16 is a diagram illustrating another embodiment of a surgical cutting block including a plurality of double cut guides and a plurality of single cut guides;

FIG. 17 is a diagram illustrating one embodiment of a surgical cutting block including a double cut guide and a plurality of single cut guides;

FIG. 18 is a diagram illustrating another embodiment of a surgical cutting block including a double cut guide and a plurality of single cut guides;

FIG. 19 is a diagram illustrating one embodiment of a surgical cutting block including a plurality of double cut guides and a single cut guide;

FIG. 20 is a diagram illustrating another embodiment of a surgical cutting block including a plurality of double cut guides and a single cut guide;

FIG. 21 is a diagram illustrating one embodiment of a surgical cutting block including a double cut guide and a plurality of single cut guides;

FIG. 22 is a diagram illustrating another embodiment of a surgical cutting block including a double cut guide and a plurality of single cut guides;

FIG. 23 is a diagram illustrating an embodiment of a surgical cutting block including a plurality of double cut guides and a single cut guide;

FIG. 24 is a diagram illustrating another embodiment of a surgical cutting block including a plurality of double cut guides and a single cut guide;

FIG. 25 is a diagram illustrating another embodiment of a surgical cutting block including a double cut guide and a plurality of single cut guides;

FIG. 26 is a diagram illustrating still another embodiment of a surgical cutting block including a double cut guide and a plurality of single cut guides;

FIG. 27 is a diagram illustrating another embodiment of a surgical cutting block including a plurality of double cut guides and a single cut guide;

FIG. 28 is a diagram illustrating still another embodiment of a surgical cutting block including a plurality of double cut guides and a single cut guide;

FIG. 29 is a diagram illustrating another embodiment of a surgical cutting block including a double cut guide and a plurality of single cut guides;

FIG. 30 is a diagram illustrating still another embodiment of a surgical cutting block including a double cut guide and a plurality of single cut guides;

FIG. 31 is a diagram illustrating another embodiment of a surgical cutting block including a plurality of double cut guides and a single cut guide;

FIG. 32 is a diagram illustrating still another embodiment of a surgical cutting block including a plurality of double cut guides and a single cut guide;

FIG. 33 is a diagram illustrating one embodiment of a surgical cutting block including a multi-cut guide and a single cut guide;

FIG. 34 is a diagram illustrating another embodiment of a surgical cutting block including a multi-cut guide and a single cut guide;

FIG. 35 is a diagram illustrating one embodiment of a surgical cutting block including a multi-cut guide and a plurality of single cut guides;

FIG. 36 is a diagram illustrating another embodiment of a surgical cutting block including a multi-cut guide and a plurality of single cut guides;

FIG. 37 is a diagram illustrating one embodiment of a surgical cutting block including a multi-cut guide and a double cut guide;

FIG. 38 is a diagram illustrating another embodiment of a surgical cutting block including a multi-cut guide and a double cut guide;

FIG. 39 is a diagram illustrating one embodiment of a surgical cutting block including a plurality of multi-cut guides;

FIG. 40A is a diagram illustrating one embodiment of a positioning fin;

FIG. 40B is a diagram illustrating a profile view of the positioning fin illustrated in FIG. 40A;

FIG. 41 is a diagram illustrating that opposite sides of a surgical cutting block can be used on the left foot and the right foot of a patient;

FIG. 42A is a diagram illustrating a profile view of one embodiment of a positioning fin and a surgical cutting board engaged with one another; and

FIG. 42B is a diagram illustrating a closer view of the positioning fin and surgical cutting board illustrated in FIG. 42A.

### DETAILED DESCRIPTION OF THE DRAWINGS

It should be understood that the language used in the present disclosure has been principally selected for readability and instructional purposes, and not to limit the scope of the subject matter disclosed herein in any manner. Further, reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including, but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

In addition, as used herein, the term “set” can mean “one or more,” unless expressly specified otherwise. The term “sets” can mean multiples of or a plurality of “one or mores,” “ones or more,” and/or “ones or mores” consistent with set theory, unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

The present technology includes surgical cutting blocks and positioning fins. Furthermore, the described features, structures, or characteristics of the various embodiments disclosed herein may be combined in any suitable manner. One skilled in the relevant art will recognize, however, that embodiments may be practiced without one or more of the specific details, or with other methods, components, materials, and so forth. In other instances, well-known structures, and/or materials are not shown or described in detail to avoid obscuring aspects of an embodiment.

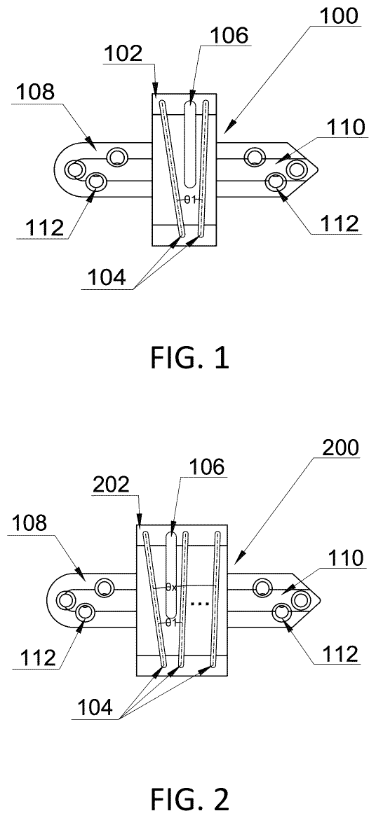

Turning now to the drawings, FIG. 1 is a diagram illustrating one embodiment of a surgical cutting block 100. At least in the illustrated embodiment, the surgical cutting block 100 includes, among other components, a cutting platform 102 (or cutting portion 102) including a plurality of single cut guides 104 (e.g., cutting slots) formed thereon, a positioning slot 106 (e.g., an aperture), a cuneiform fixation platform 108, a metatarsal fixation platform 110, and a plurality of wire holes 112.

The cutting platform 102 may be formed of any suitable material. In certain embodiments, the material included in and/or forming the cutting platform 102 is sterilizable.

In the embodiment illustrated in FIG. 1, the plurality of single cut guides 104 includes a single cut guide 104 located on opposite sides of the positioning slot 106. Other embodiments may include the plurality of single cut guides 104 positioned at other locations on the cutting platform 102 (e.g., on the same side of the positioning slot 106, among other locations that are possible and contemplated herein). That is, the plurality of single cut guides 104 may be positioned/formed at any suitable location on the cutting platform 102.

The single cut guides 104 each include a size and/or width capable of accepting a surgical cutting tool (e.g., a scalpel, surgical knife, etc.) and provide a single path for the surgical cutting tool to follow while the surgical cutting tool is being used to produce an incision in a patient. Each single cut guide 104 includes an angle (θ), which can be any suitable angle (e.g., an angle (θ) in the range of about one degree (1°) to about 90 degrees (90°)) with respect to any suitable reference point that can provide a predetermined and/or target path for the surgical cutting tool to follow while the surgical cutting tool is cutting a patient and/or performing a surgical procedure.

In some embodiments, all of the single cut guides 104 are angled in the same direction (e.g., angled toward the left or toward the right with respect to the user (e.g., a medical professional)). In the illustrated embodiment, all of the single cut guides 104 are angled in the different directions (see, e.g., FIG. 1).

In additional or alternative embodiments, a pair of single cut guides 104 are positioned on the cutting platform 102 such that there is an angle (θ₁) created between the pair of single cut guides 104, which may be any suitable angle (θ₁). In various embodiments, the angle (θ₁) is in the range of about fifteen degrees (15°) to about 75 degrees (75°).

The positioning slot 106 is configured to facilitate positioning the surgical cutting block 100 over a target surgical area. The positioning slot 106 may include any suitable size and/or shape that can facilitate positioning the surgical cutting block 100 over a target surgical area.

A cuneiform fixation platform 108 may include any suitable size, shape, and/or material(s) that can facilitate fixating the surgical cutting block 100 to a cuneiform. In certain embodiments, the cuneiform fixation platform 108 is located on a side of the cutting platform 102, among other locations that are possible and contemplated herein.

A metatarsal fixation platform 110 may include any suitable size, shape, and/or material(s) that can facilitate fixating the surgical cutting block 100 to a metatarsal. In various embodiments, the metatarsal fixation platform 110 is located on a side of the cutting platform 102, among other locations that are possible and contemplated herein. In some embodiments, the cuneiform fixation platform 108 is located on one side of the cutting platform 102 and the metatarsal fixation platform 110 is located on the other side of the cutting platform 102 opposite the cuneiform fixation platform 108, among other locations and/or relative positions that are possible and contemplated herein.

In some embodiments, the cuneiform fixation platform 108 and the metatarsal fixation platform 110 include a set of wire holes 112 formed respectively therein. Each of the he cuneiform fixation platform 108 and the metatarsal fixation platform 110 include one or more wire holes 112 for attaching the surgical cutting block 100 to a cuneiform and a metatarsal, respectively, which can be any suitable quantity of wire holes 112. Further, the wire holes 112 may include any suitable size and/or shape that can facilitate fixating the surgical cutting block 100 to a cuneiform and a metatarsal via a pin passing through the wire holes 112 on the cuneiform fixation platform 108 and the metatarsal fixation platform 110, respectively.

FIG. 2 is a diagram illustrating another embodiment of a surgical cutting block 200 including a plurality of single cut guides 104. In this embodiment, a sub-plurality of single cut guides 104 are positioned/located on a cutting platform 202 on the side of the positioning slot 106 toward the metatarsal fixation platform 110. Further, surgical cutting block 200 includes one single cut guide 104 positioned/located on the cutting platform 202 on the side of the positioning slot 106 toward the cuneiform fixation platform 108.

In some embodiments, all of the single cut guides 104 are angled in the same direction (e.g., angled toward the left or toward the right with respect to the user (e.g., a medical professional)). In the illustrated embodiment, all of the single cut guides 104 on the same side of the positioning slot 106 are angled in the same direction and in a different direction than the single cut guide 104 on the other side of the positioning slot 106.

In additional or alternative embodiments, the single cut guides 104 are positioned on the cutting platform 202 such that there is an angle (θ₁) and (θₓ) created between the sub-plurality of single cut guides 104 and the one single cut guide 104, which may be any suitable angle. In various embodiments, the respective angles (θ₁) and (θₓ) may each be in the range of about fifteen degrees (15°) to about 75 degrees (75°).

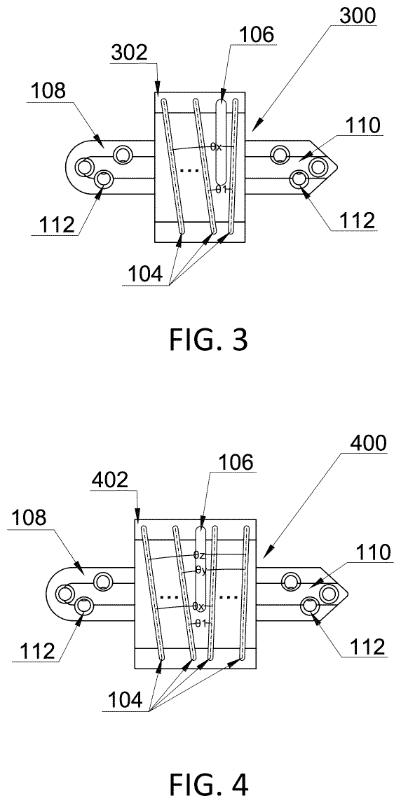

FIG. 3 is a diagram illustrating yet another embodiment of a surgical cutting block 300 including a plurality of single cut guides 104. In this embodiment, a sub-plurality of single cut guides 104 are positioned/located on a cutting platform 302 on the side of the positioning slot 106 toward the cuneiform fixation platform 108. Further, surgical cutting block 300 includes one single cut guide 104 positioned/located on the cutting platform 302 on the side of the positioning slot 106 toward the metatarsal fixation platform 110.

In some embodiments, all of the single cut guides 104 are angled in the same direction (e.g., angled toward the left or toward the right with respect to the user (e.g., a medical professional)). In the illustrated embodiment, all of the single cut guides 104 on the same side of the positioning slot 106 are angled in the same direction and in a different direction than the single cut guide 104 on the other side of the positioning slot 106.

In additional or alternative embodiments, the single cut guides 104 are positioned on the cutting platform 302 such that there is an angle (θ₁) and (θₓ) created between the sub-plurality of single cut guides 104 and the one single cut guide 104, which may be any suitable angle. In various embodiments, the respective angles (θ₁) and (θₓ) may each be in the range of about 15° to about 75°.

FIG. 4 is a diagram illustrating still another embodiment of a surgical cutting block 400 including a plurality of single cut guides 104. In this embodiment, a sub-plurality of single cut guides 104 are positioned/located on a cutting platform 402 on the side of the positioning slot 106 toward the metatarsal fixation platform 110. Further, surgical cutting block 400 includes a sub-plurality of single cut guides 104 positioned/located on the cutting platform 102 on the side of the positioning slot 106 toward the cuneiform fixation platform 108.

In some embodiments, all of the single cut guides 104 are angled in the same direction (e.g., angled toward the left or toward the right with respect to the user (e.g., a medical professional)). In the illustrated embodiment, all of the single cut guides 104 on the same side of the positioning slot 106 are angled in the same direction and in a different direction than the single cut guides 104 on the other side of the positioning slot 106.

In additional or alternative embodiments, the single cut guides 104 are positioned on the cutting platform 402 such that there is an angle (θ₁), (θₓ), (θ_{y}), and (θ_{z}) created between the sub-plurality of single cut guides 104 located on each side of the positioning slot 106, which may be any suitable angle. In various embodiments, the respective angles (θ₁), (θₓ), (θ_{y}), and (θ_{z}) may each be in the range of about 15° to about 75°.

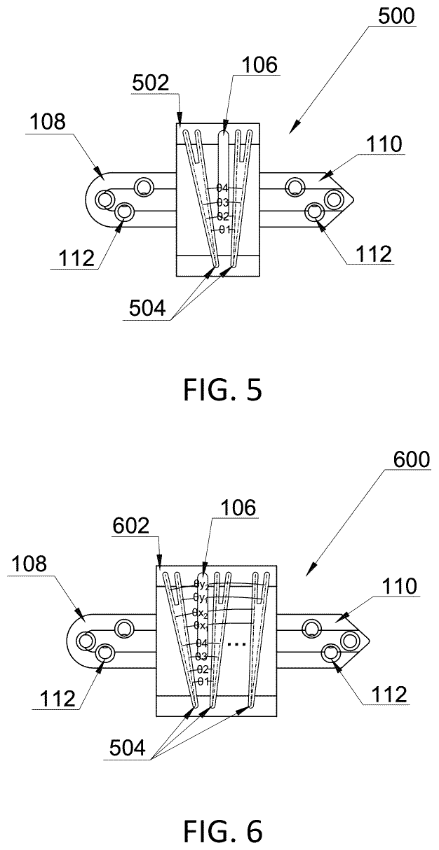

FIG. 5 is a diagram illustrating one embodiment of a surgical cutting block 500 including a plurality of double cut guides 104. In this embodiment, a double cut guide 504 is positioned/located on a cutting platform 502 on each side of the positioning slot 106, among other positions that are possible and contemplated herein.

The double cut guides 504 each include a pair of paths that can include any suitable size and/or width capable of accepting a surgical cutting tool (e.g., a scalpel, surgical knife, etc.) and provide a respective path for the surgical cutting tool to follow while the surgical cutting tool is being used to produce an incision in a patient. Each double cut guide 504 includes an angle, which can be any suitable angle (e.g., an angle in the range of about one degree (1°) to about 90 degrees (90°)) with respect to any suitable reference point that can provide a predetermined and/or target path for the surgical cutting tool to follow while the surgical cutting tool is cutting a patient and/or performing a surgical procedure.

In some embodiments, all of the double cut guides 504 are angled in the same direction (e.g., angled toward the left or toward the right with respect to the user (e.g., a medical professional)). In the illustrated embodiment, all of the double cut guides 504 are angled in the different directions.

In additional or alternative embodiments, a pair of double cut guides 504 are positioned on the cutting platform 502 such that there is an angle (θ₁), (θ₂), (θ₃), and (θ₄) created between the double cut guides 504 located on each side of the positioning slot 106, which may be any suitable angle. In various embodiments, the respective angles (θ₁), (θ₂), (θ₃), and (θ₄) may each be in the range of about 15° to about 75°.

FIG. 6 is a diagram illustrating another embodiment of a surgical cutting block 600 including a plurality of double cut guides 504. In this embodiment, a sub-plurality of double cut guides 504 are positioned/located on a cutting platform 602 on the side of the positioning slot 106 toward the metatarsal fixation platform 110. Further, surgical cutting block 600 includes one double cut guide 504 positioned/located on the cutting platform 602 on the side of the positioning slot 106 toward the cuneiform fixation platform 108.

In some embodiments, all of the double cut guides 504 are angled in the same direction (e.g., angled toward the left or toward the right with respect to the user (e.g., a medical professional)). In the illustrated embodiment, all of the double cut guides 504 on the same side of the positioning slot 106 are angled in the same direction and in a different direction than the double cut guide 504 on the other side of the positioning slot 106.

In additional or alternative embodiments, the double cut guides 504 are positioned on the cutting platform 602 such that there is an (θ₁), (θ2), (θ3), (θ₄), (θₓ₁), (θₓ₂), (θ_{y1}), and (θ_{y2}) created between the double cut guides 504 located on each side of the positioning slot 106, which may be any suitable angle. In various embodiments, the respective angles (θ₁), (θ₂), (θ₃), (θ₄), (θₓ), (θₓ₂), (θ_{y1}), and (θ_{y2}) may each be in the range of about 15° to about 75°.

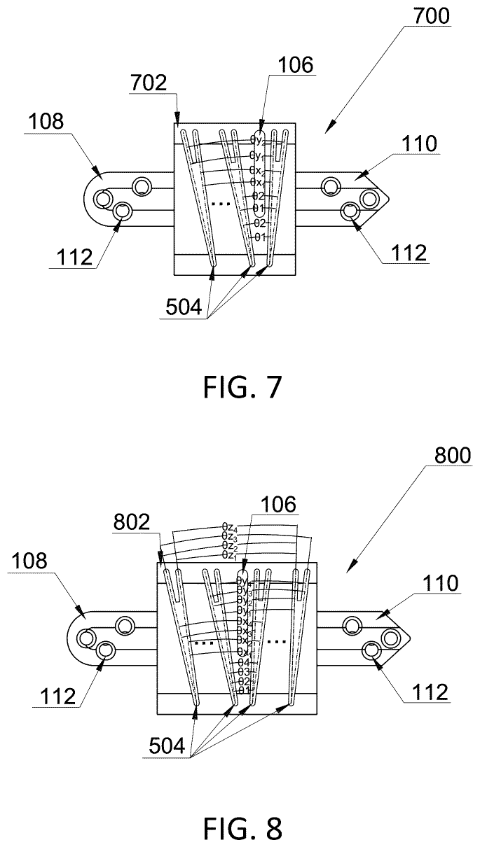

FIG. 7 is a diagram illustrating another embodiment of a surgical cutting block 700 including a plurality of double cut guides 504. In this embodiment, a sub-plurality of double cut guides 504 are positioned/located on a cutting platform 702 on the side of the positioning slot 106 toward the cuneiform fixation platform 108. Further, surgical cutting block 700 includes one double cut guide 504 positioned/located on the cutting platform 702 on the side of the positioning slot 106 toward the metatarsal fixation platform 110.

In some embodiments, all of the double cut guides 504 are angled in the same direction (e.g., angled toward the left or toward the right with respect to the user (e.g., a medical professional)). In the illustrated embodiment, all of the double cut guides 504 on the same side of the positioning slot 106 are angled in the same direction and in a different direction than the double cut guide 504 on the other side of the positioning slot 106.

In additional or alternative embodiments, the double cut guides 504 are positioned on the cutting platform 702 such that there is an (θ₁), (θ2), (θ3), (θ₄), (θₓ₁), (θₓ₂), (θ_{y1}), and (θ_{y2}) created between the double cut guides 504 located on each side of the positioning slot 106, which may be any suitable angle. In various embodiments, the respective angles (θ₁), (θ₂), (θ₃), (θ₄), (θₓ₁), (θₓ₂), (θ_{y1}), and (θ_{y2}) may each be in the range of about 15° to about 75°.

FIG. 8 is a diagram illustrating another embodiment of a surgical cutting block 800 including a plurality of double cut guides 504. In this embodiment, a sub-plurality of double cut guides 504 are positioned/located on a cutting platform 802 on both sides of the positioning slot.

In some embodiments, all of the double cut guides 504 are angled in the same direction (e.g., angled toward the left or toward the right with respect to the user (e.g., a medical professional)). In the illustrated embodiment, all of the double cut guides 504 on the same side of the positioning slot 106 are angled in the same direction and in a different direction than the double cut guides 504 on the other side of the positioning slot 106.

In additional or alternative embodiments, the double cut guides 504 are positioned on the cutting platform 802 such that there is an angle (θ₁), (θ₂), (θ₃), (θ₄), (θₓ₁), (θₓ₂), (θₓ₃), (θₓ₄), (θ_{y1}), (θ_{y2}), (θ_{y3}), and (θ_{y4}) created between the double cut guides 504 located on each side of the positioning slot 106, which may be any suitable angle. In various embodiments, the respective angles θ₁), (θ₂), (θ₃), (θ₄), (θₓ₁), (θₓ₂), (θₓ₃), (θₓ₄), (θ_{y1}), (θ_{y2}), (θ_{y3}), and (θ_{y4}) may each be in the range of about 15° to about 75°.

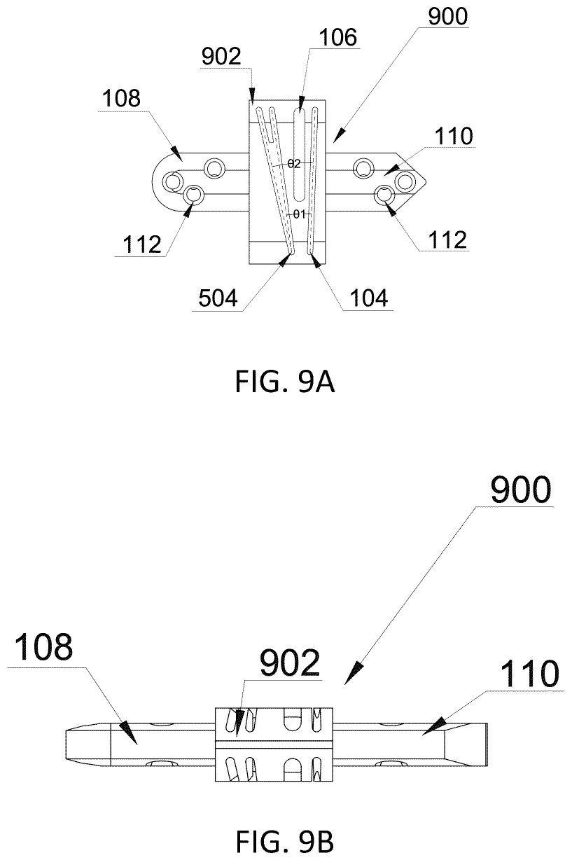

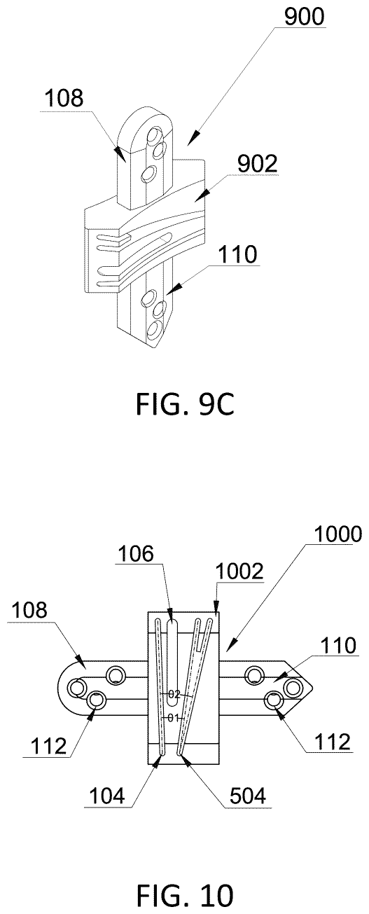

FIG. 9A is a diagram illustrating one embodiment of a surgical cutting block 900 including a single cut guide 104 and a double cut guide 504. In this embodiment, the single cut guide 104 is positioned/located on a cutting platform 902 on the side of the positioning slot 106 toward the metatarsal fixation platform 110. Further, surgical cutting block 900 includes the double cut guide 504 positioned/located on the cutting platform 902 on the side of the positioning slot 106 toward the cuneiform fixation platform 108.

In some embodiments, the single cut guide 104 and the double cut guide 504 are angled in the same direction (e.g., angled toward the left or toward the right with respect to the user (e.g., a medical professional)). In the illustrated embodiment, the single cut guide 104 and the double cut guide 504 are angled in different directions.

In additional or alternative embodiments, the single cut guide 104 and the double cut guide 504 are positioned on the cutting platform 902 such that there is an angle (θ₁) and (θ₂) created between the single cut guide 104 and the double cut guide 504, which may be any suitable angle. In various embodiments, the respective angles (θ₁) and (θ₂) may each be in the range of about 15° to about 75°.

FIG. 9B is diagram illustrating a side profile of the surgical cutting block 900. FIG. 9B illustrates that a single cut guide 104 (e.g., an aperture) and a double cut guide 504 (e.g., an aperture) extend through the cutting platform 902.

FIG. 9C is a diagram illustrating side view of the surgical cutting block 900. FIG. 9C illustrates that the cutting platform 902 may include a slope and/or sloped portion that can be any suitable slope included at any suitable portion of the cutting platform.

FIG. 10 is a diagram illustrating another embodiment of a surgical cutting block 1000 including a single cut guide 104 and a double cut guide 504. In this embodiment, the single cut guide 104 is positioned/located on a cutting platform 1002 on the side of the positioning slot 106 toward the cuneiform fixation platform 108. Further, surgical cutting block 1000 includes the double cut guide 504 positioned/located on the cutting platform 1002 on the side of the positioning slot 106 toward the metatarsal fixation platform 110.

In some embodiments, the single cut guide 104 and the double cut guide 504 are angled in the same direction (e.g., angled toward the left or toward the right with respect to the user (e.g., a medical professional)). In the illustrated embodiment, the single cut guide 104 and the double cut guide 504 are angled in different directions.

In additional or alternative embodiments, the single cut guide 104 and the double cut guide 504 are positioned on the cutting platform 102 such that there is an angle (θ₁) and (θ₂) created between the single cut guide 104 and the double cut guide 504, which may be any suitable angle. In various embodiments, the respective angles (θ₁) and (θ₂) may each be in the range of about 15° to about 75°.

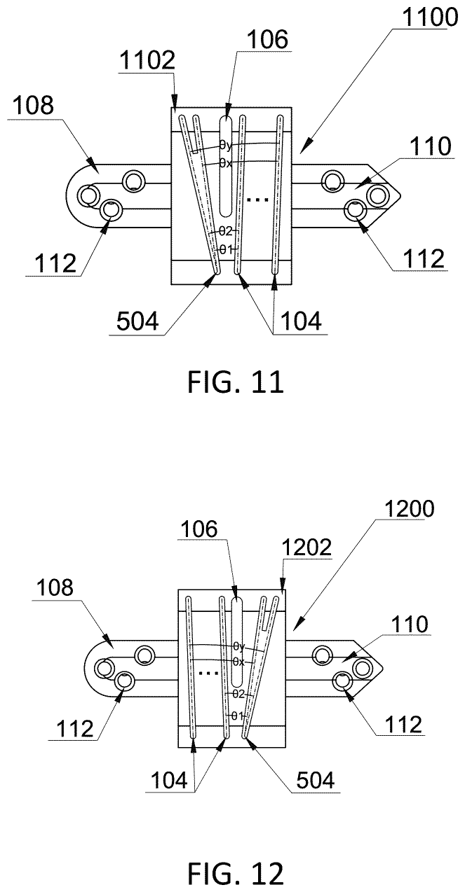

FIG. 11 is a diagram illustrating one embodiment of a surgical cutting block 1100 including a plurality of single cut guides 104 and a double cut guide 504. In this embodiment, the plurality of single cut guides 104 are positioned/located on a cutting platform 1102 on the side of the positioning slot 106 toward the metatarsal fixation platform 110. Further, surgical cutting block 1100 includes the double cut guide 504 positioned/located on the cutting platform 1102 on the side of the positioning slot 106 toward the cuneiform fixation platform 108.

In some embodiments, the plurality of single cut guides 104 and the double cut guide 504 are angled in the same direction (e.g., angled toward the left or toward the right with respect to the user (e.g., a medical professional)). In the illustrated embodiment, the plurality of single cut guides 104 and the double cut guide 504 are angled in different directions.

In additional or alternative embodiments, the plurality of single cut guides 104 and the double cut guide 504 are positioned on the cutting platform 1102 such that there is an angle (θ₁), (θ₂), (θₓ), and (θ_{y}) created between the plurality of single cut guides 104 and the double cut guide 504, which may be any suitable angle. In various embodiments, the respective angles (θ₁), (θ₂), (θₓ), and (θ_{y}) may each be in the range of about 15° to about 75°.

FIG. 12 is a diagram illustrating another embodiment of a surgical cutting block including a plurality of single cut guides 104 and a double cut guide 504. In this embodiment, the plurality of single cut guides 104 are positioned/located on a cutting platform 1202 on the side of the positioning slot 106 toward the cuneiform fixation platform 108. Further, surgical cutting block 1200 includes the double cut guide 504 positioned/located on the cutting platform 1202 on the side of the positioning slot 106 toward the metatarsal fixation platform 110.

In some embodiments, the plurality of single cut guides 104 and the double cut guide 504 are angled in the same direction (e.g., angled toward the left or toward the right with respect to the user (e.g., a medical professional)). In the illustrated embodiment, the plurality of single cut guides 104 and the double cut guide 504 are angled in different directions.

In additional or alternative embodiments, the plurality of single cut guides 104 and the double cut guide 504 are positioned on the cutting platform 1202 such that there is an angle (θ₁), (θ₂), (θₓ), and (θ_{y}) created between the plurality of single cut guides 104 and the double cut guide 504, which may be any suitable angle. In various embodiments, the respective angles (θ₁), (θ₂), (θₓ), and (θ_{y}) may each be in the range of about 15° to about 75°.

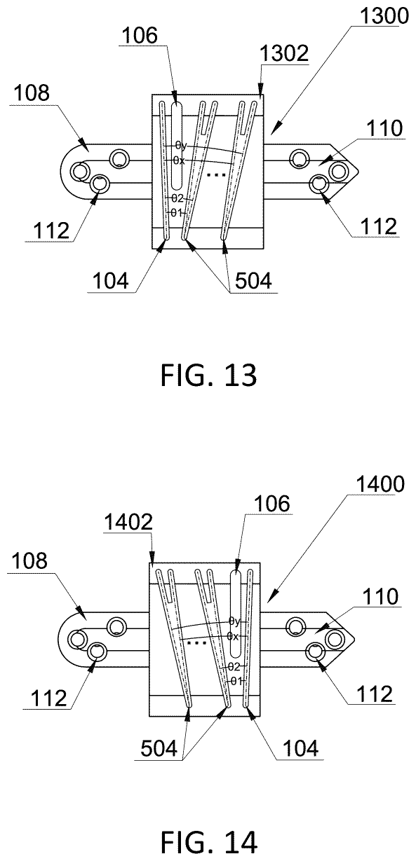

FIG. 13 is a diagram illustrating one embodiment of a surgical cutting block 1300 including a plurality of double cut guides 504 and a single cut guide 104. In this embodiment, the plurality of double cut guides 504 are positioned/located on a cutting platform 1302 on the side of the positioning slot 106 toward the metatarsal fixation platform 110. Further, surgical cutting block 1300 includes the single cut guide 104 positioned/located on the cutting platform 1302 on the side of the positioning slot 106 toward the cuneiform fixation platform 108.

In some embodiments, the plurality of double cut guides 504 and the single cut guide 104 are angled in the same direction (e.g., angled toward the left or toward the right with respect to the user (e.g., a medical professional)). In the illustrated embodiment, the plurality of double cut guides 504 and the single cut guide 104 are angled in different directions.

In additional or alternative embodiments, the plurality of double cut guides 504 and the single cut guide 104 are positioned on the cutting platform 1302 such that there is an angle (θ₁), (θ₂), (θₓ), and (θ_{y}) created between the single cut guide 104 and the plurality of double cut guides 504, which may be any suitable angle. In various embodiments, the respective angles (θ₁), (θ₂), (θₓ), and (θ_{y}) may each be in the range of about 15° to about 75°.

FIG. 14 is a diagram illustrating another embodiment of a surgical cutting block 1400 including a plurality of double cut guides 504 and a single cut guide 104. In this embodiment, the plurality of double cut guides 504 are positioned/located on a cutting platform 1402 on the side of the positioning slot 106 toward the cuneiform fixation platform 108. Further, surgical cutting block 1400 includes the single cut guide 104 positioned/located on the cutting platform 1402 on the side of the positioning slot 106 toward the metatarsal fixation platform 110.

In some embodiments, the plurality of double cut guides 504 and the single cut guide 104 are angled in the same direction (e.g., angled toward the left or toward the right with respect to the user (e.g., a medical professional)). In the illustrated embodiment, the plurality of double cut guides 504 and the single cut guide 104 are angled in different directions.

In additional or alternative embodiments, the plurality of double cut guides 504 and the single cut guide 104 are positioned on the cutting platform 1402 such that there is an angle (θ₁), (θ₂), (θₓ), and (θ_{y}) created between the single cut guide 104 and the plurality of double cut guides 504, which may be any suitable angle. In various embodiments, the respective angles (θ₁), (θ₂), (θₓ), and (θ_{y}) may each be in the range of about 15° to about 75°.

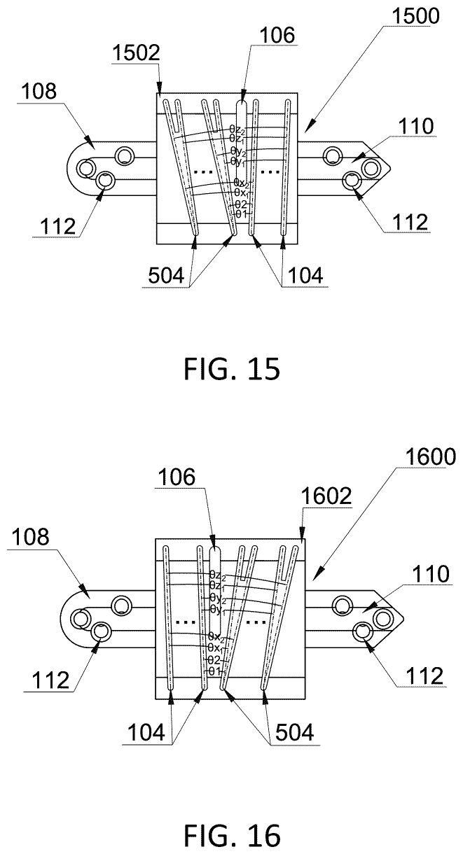

FIG. 15 is a diagram illustrating one embodiment of a surgical cutting block 1500 including a plurality of double cut guides 504 and a plurality of single cut guides 104. In this embodiment, the plurality of double cut guides 504 are located/positioned on a cutting platform 1502 toward the cuneiform fixation platform 108 and the plurality of single cut guides 104 are located/positioned on the cutting platform 1502 toward the metatarsal fixation platform 110.

In some embodiments, the plurality of double cut guides 504 and the plurality of single cut guides 104 are angled in the same direction (e.g., angled toward the left or toward the right with respect to the user (e.g., a medical professional)). In the illustrated embodiment, the plurality of double cut guides 504 and the plurality of single cut guides 104 are angled in different directions.

In additional or alternative embodiments, the plurality of double cut guides 504 and the plurality of single cut guides 104 are positioned on the cutting platform 1402 such that there is an angle (θ₁), (θ₂), (θₓ₁), (θₓ₂), (θ_{y1}), (θ_{y2}), (θ_{z1}), and (θ_{z2}) created between the plurality of single cut guides 104 and the plurality of double cut guides 504, which may be any suitable angle. In various embodiments, the respective angles (θ₁), (θ₂), (θₓ₁), (θₓ₂), (θ_{y1}), (θ_{y2}), (θ_{z1}), and (θ_{z2}) may each be in the range of about 15° to about 75°.

FIG. 16 is a diagram illustrating another embodiment of a surgical cutting block 1600 including a plurality of double cut guides 504 and a plurality of single cut guides 104. In this embodiment, the plurality of double cut guides 504 are located/positioned on a cutting platform 1602 toward the metatarsal fixation platform 110 and the plurality of single cut guides 104 are located/positioned on the cutting platform 1602 toward the cuneiform fixation platform 108.

In some embodiments, the plurality of double cut guides 504 and the plurality of single cut guides 104 are angled in the same direction (e.g., angled toward the left or toward the right with respect to the user (e.g., a medical professional)). In the illustrated embodiment, the plurality of double cut guides 504 and the plurality of single cut guides 104 are angled in different directions.

In additional or alternative embodiments, the plurality of double cut guides 504 and the plurality of single cut guides 104 are positioned on the cutting platform 1402 such that there is an angle (θ₁), (θ₂), (θₓ₁), (θₓ₂), (θ_{y1}), (θ_{y2}), (θ_{z1}), and (θ_{z2}) created between the plurality of single cut guides 104 and the plurality of double cut guides 504, which may be any suitable angle. In various embodiments, the respective angles (θ₁), (θ₂), (θₓ₁), (θₓ₂), (θ_{y1}), (θ_{y2}), (θ_{z1}), and (θ_{z2}) may each be in the range of about 15° to about 75°.

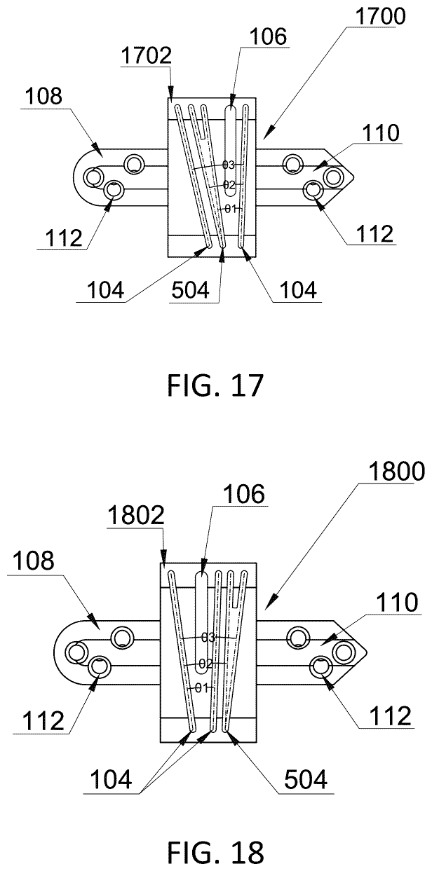

FIG. 17 is a diagram illustrating one embodiment of a surgical cutting block 1700 including a double cut guide 504 and a plurality of single cut guides 104. In this embodiment, the double cut guide 504 and one single cut guide 104 are located/positioned on a cutting platform 1702 toward the cuneiform fixation platform 108 and one single cut guide 104 is located/positioned on the cutting platform 1702 toward the metatarsal fixation platform 110. Further, the double cut guide 504 is located/positioned to the right of the single cut guide 104 on the cuneiform fixation platform 108 side of the cutting platform 102.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the single cut guide 104 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), and (θ₃) created between the double cut guide 504 and the single cut guide 104 and the other single cut guide 104, which may be any suitable angle. In various embodiments, the respective angles (θ₁), (θ₂), and (θ₃) may each be in the range of about 15° to about 75°.

FIG. 18 is a diagram illustrating another embodiment of a surgical cutting block 1800 including a double cut guide 504 and a plurality of single cut guides 104. In this embodiment, the double cut guide 504 and one single cut guide 104 are located/positioned on a cutting platform 1802 toward the metatarsal fixation platform 110 and one single cut guide 104 is located/positioned on the cutting platform 1802 toward the cuneiform fixation platform 108. Further, the double cut guide 504 is located/positioned to the right of the single cut guide 104 on the metatarsal fixation platform 110 side of the cutting platform 1802.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the single cut guide 104 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), and (θ₃) created between the double cut guide 504 and the single cut guide 104 and the other single cut guide 104, which may be any suitable angle. In various embodiments, the respective angles (θ₁), (θ₂), and (θ₃) may each be in the range of about 15° to about 75°.

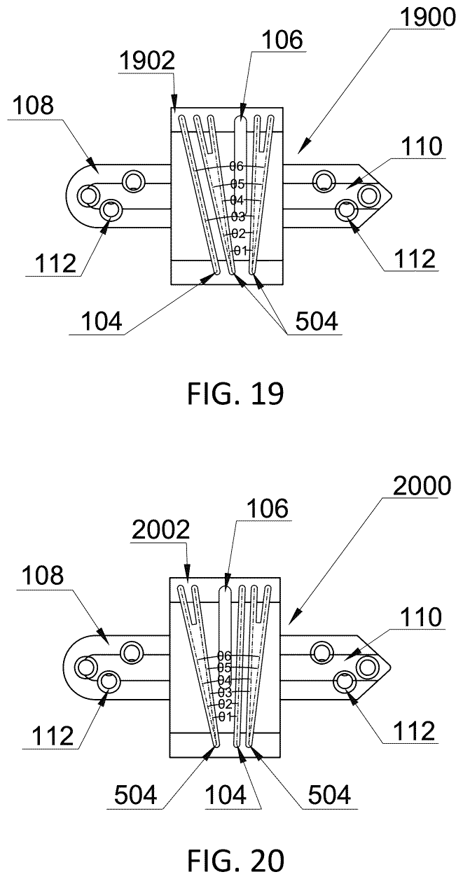

FIG. 19 is a diagram illustrating one embodiment of a surgical cutting block 1900 including a plurality of double cut guides 504 and a single cut guide 104. In this embodiment, one double cut guide 504 and the single cut guide 104 are located/positioned on a cutting platform 1902 toward the cuneiform fixation platform 108 and one double cut guide 504 is located/positioned on the cutting platform 1902 toward the metatarsal fixation platform 110. Further, the double cut guide 504 is located/positioned to the right of the single cut guide 104 on the cuneiform fixation platform 108 side of the cutting platform 1902.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the double cut guide 504 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), and (θ₆) created between the double cut guide 504 and the single cut guide 104 and the other double cut guide 504, which may be any suitable angle. In various embodiments, the respective angles (θ₁), (θ₂), (θ₃), (θ₄), (θ₅), and (θ₆) may each be in the range of about 15° to about 75°.

FIG. 20 is a diagram illustrating another embodiment of a surgical cutting block 2000 including a plurality of double cut guides 504 and a single cut guide 104. In this embodiment, one double cut guide 504 and the single cut guide 104 are located/positioned on a cutting platform 2002 toward the metatarsal fixation platform 110 and one double cut guide 504 is located/positioned on the cutting platform 2002 toward the cuneiform fixation platform 108. Further, the double cut guide 504 is located/positioned to the right of the single cut guide 104 on the metatarsal fixation platform 110 side of the cutting platform 2002.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the double cut guide 504 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), and (θ₆) created between the double cut guide 504 and the single cut guide 104 and the other double cut guide 504, which may be any suitable angle. In various embodiments, the respective angles (θ₁), (θ₂), (θ₃), (θ₄), (θ₅), and (θ₆) may each be in the range of about 15° to about 75°.

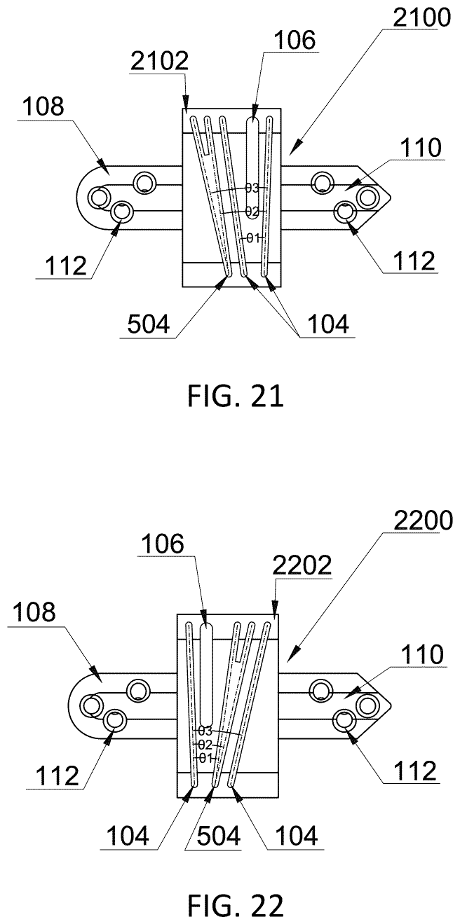

FIG. 21 is a diagram illustrating one embodiment of a surgical cutting block 2100 including a double cut guide 504 and a plurality of single cut guides 104. In this embodiment, the double cut guide 504 and one single cut guide 104 are located/positioned on a cutting platform 2102 toward the cuneiform fixation platform 108 and one single cut guide 104 is located/positioned on the cutting platform 2102 toward the metatarsal fixation platform 110. Further, the double cut guide 504 is located/positioned to the left of the single cut guide 104 on the cuneiform fixation platform 108 side of the cutting platform 2102.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the single cut guide 104 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), and (θ₃) created between the double cut guide 504 and the single cut guide 104 and the other single cut guide 104, which may be any suitable angle. In various embodiments, the respective angles (θ₁), (θ₂), and (θ₃) may each be in the range of about 15° to about 75°.

FIG. 22 is a diagram illustrating another embodiment of a surgical cutting block 2200 including a double cut guide 504 and a plurality of single cut guides 104. In this embodiment, the double cut guide 504 and one single cut guide 104 are located/positioned on a cutting platform 2202 toward the metatarsal fixation platform 110 and one single cut guide 104 is located/positioned on the cutting platform 2202 toward the cuneiform fixation platform 108. Further, the double cut guide 504 is located/positioned to the left of the single cut guide 104 on the metatarsal fixation platform 110 side of the cutting platform 2202.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the single cut guide 104 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), and (θ₃) created between the double cut guide 504 and the single cut guide 104 and the other single cut guide 104, which may be any suitable angle. In various embodiments, the respective angles (θ₁), (θ₂), and (θ₃) may each be in the range of about 15° to about 75°.

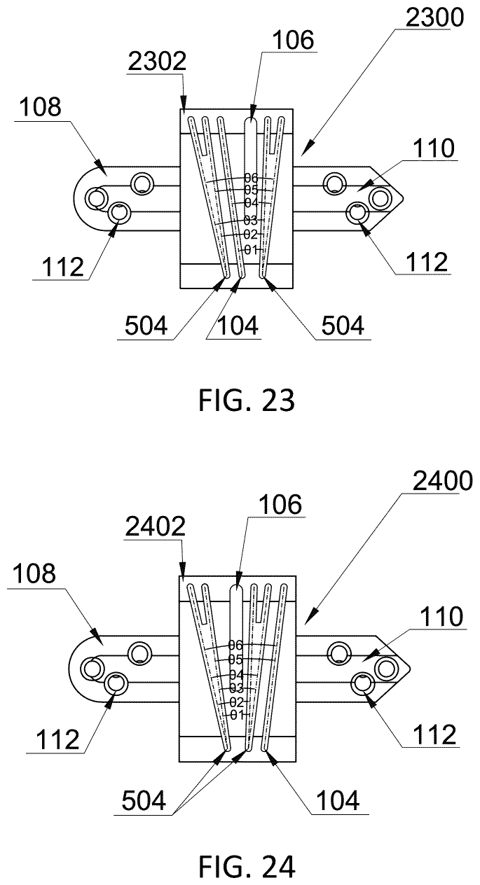

FIG. 23 is a diagram illustrating one embodiment of a surgical cutting block 2300 including a plurality of double cut guides 504 and a single cut guide 104. In this embodiment, one double cut guide 504 and the single cut guide 104 are located/positioned on a cutting platform 2302 toward the cuneiform fixation platform 108 and one double cut guide 504 is located/positioned on the cutting platform 2302 toward the metatarsal fixation platform 110. Further, the double cut guide 504 is located/positioned to the left of the single cut guide 104 on the cuneiform fixation platform 108 side of the cutting platform 2302.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the double cut guide 504 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), and (θ₆) created between the double cut guide 504 and the single cut guide 104 and the other double cut guide 504, which may be any suitable angle. In various embodiments, the respective angles (θ₁), (θ₂), (θ₃), (θ₄), (θ₅), and (θ₆) may each be in the range of about 15° to about 75°.

FIG. 24 is a diagram illustrating another embodiment of a surgical cutting block 2400 including a plurality of double cut guides 504 and a single cut guide 104. In this embodiment, one double cut guide 504 and the single cut guide 104 are located/positioned on a cutting platform 2402 toward the metatarsal fixation platform 110 and one double cut guide 504 is located/positioned on the cutting platform 2402 toward the cuneiform fixation platform 108. Further, the double cut guide 504 is located/positioned to the left of the single cut guide 104 on the metatarsal fixation platform 110 side of the cutting platform 2402.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the double cut guide 504 on the opposite side of the positioning slot 106 is angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), and (θ₆) created between the double cut guide 504 and the single cut guide 104 and the other double cut guide 504, which may be any suitable angle. In various embodiments, the respective angles (θ₁), (θ₂), (θ₃), (θ₄), (θ₅), and (θ₆) may each be in the range of about 15° to about 75°.

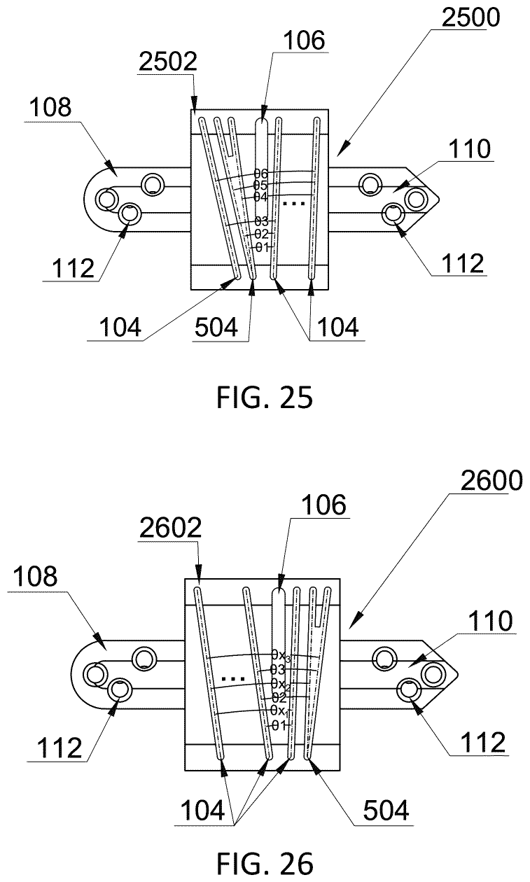

FIG. 25 is a diagram illustrating another embodiment of a surgical cutting block 2500 including a double cut guide 504 and a plurality of single cut guides 104. In this embodiment, the double cut guide 504 and one single cut guide 104 are located/positioned on a cutting platform 2502 toward the cuneiform fixation platform 108 and a plurality of single cut guides 104 are located/positioned on the cutting platform 2502 toward the metatarsal fixation platform 110. Further, the double cut guide 504 is located/positioned to the right of the single cut guide 104 on the cuneiform fixation platform 108 side of the cutting platform 2502.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the plurality of single cut guides 104 on the opposite side of the positioning slot 106 are angled in the direction as one another and a different direction than the double cut guide 504 and the single cut guide 104.

In additional or alternative embodiments, the double cut guide 504 and the single cut guide 104 are positioned on the cutting platform 1402 such that there is an angle (θ₁), (θ₂), (θ₃), (θ₄), (θ₅), and (θ₆) created between the double cut guide 504 and the single cut guide 104 and the other plurality of single cut guides 104, which may be any suitable angle. In various embodiments, the respective angles (θ₁), (θ₂), (θ₃), (θ₄), (θ₅), and (θ₆) may each be in the range of about 15° to about 75°.

FIG. 26 is a diagram illustrating another embodiment of a surgical cutting block 2600 including a double cut guide 504 and a plurality of single cut guides 104. In this embodiment, the double cut guide 504 and one single cut guide 104 are located/positioned on a cutting platform 2602 toward the metatarsal fixation platform 110 and a plurality of single cut guides 104 are located/positioned on the cutting platform 2602 toward the cuneiform fixation platform 108. Further, the double cut guide 504 is located/positioned to the right of the single cut guide 104 on the metatarsal fixation platform 110 side of the cutting platform 2602.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction, the plurality of single cut guides 104 on the opposite side of the positioning slot 106 are angled in the direction as one another and a different direction than the double cut guide 504 and the single cut guide 104.

In additional or alternative embodiments, the double cut guide 504 and the single cut guide 104 are positioned on the cutting platform 1402 such that there is an angle (θ₁), (θ₂), (θ₃), (θₓ₁), (θₓ₂), and (θₓ₃) created between the double cut guide 504 and the single cut guide 104 and the other plurality of single cut guides 104, which may be any suitable angle. In various embodiments, the respective angles (θ₁), (θ₂), (θ₃), (θₓ₁), (θₓ₂), and (θₓ₃) may each be in the range of about 15° to about 75°.

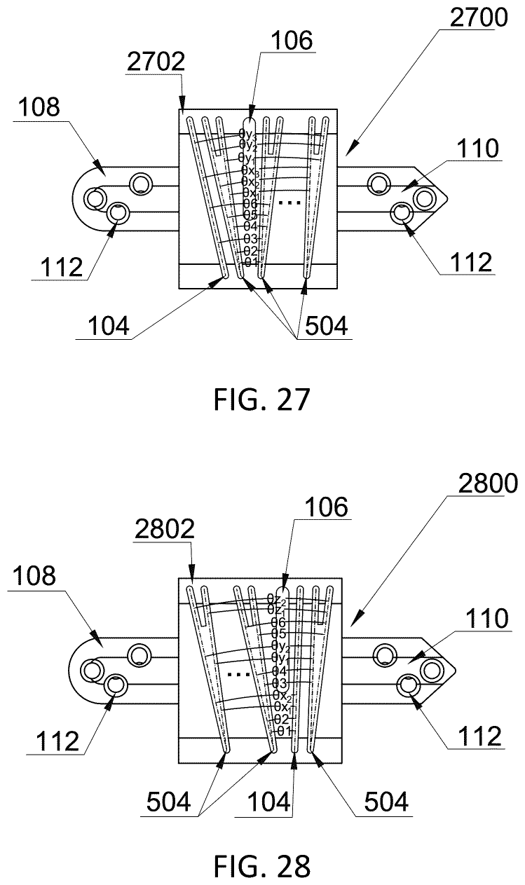

FIG. 27 is a diagram illustrating one embodiment of a surgical cutting block 2700 including a plurality of double cut guides 504 and a single cut guide 104. In this embodiment, one double cut guide 504 and the single cut guide 104 are located/positioned on a cutting platform 2702 toward the cuneiform fixation platform 108 and a plurality of double cut guides 504 are located/positioned on the cutting platform 2702 toward the metatarsal fixation platform 110. Further, the double cut guide 504 is located/positioned to the right of the single cut guide 104 on the cuneiform fixation platform 108 side of the cutting platform 2702.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the plurality of double cut guides 504 on the opposite side of the positioning slot 106 are angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), (θ₆), (θₓ₁), (θₓ₂), (θₓ₃), (θ_{y1}), (θ_{y2}), and (θ_{y3}) created between the double cut guide 504 and the single cut guide 104 and the plurality of double cut guides 504, which may be any suitable angle. In various embodiments, the respective angles (θ₁), (θ₂), (θ₃), (θ₄), (θ₅), (θ₆), (θₓ₁), (θₓ₂), (θₓ₃), (θ_{y1}), (θ_{y2}), and (θ_{y3}) may each be in the range of about 15° to about 75°.

FIG. 28 is a diagram illustrating another embodiment of a surgical cutting block 2800 including a plurality of double cut guides 504 and a single cut guide 104. In this embodiment, one double cut guide 504 and the single cut guide 104 are located/positioned on a cutting platform 2802 toward the metatarsal fixation platform 110 and a plurality of double cut guides 504 are located/positioned on the cutting platform 2802 toward the cuneiform fixation platform 108. Further, the double cut guide 504 is located/positioned to the right of the single cut guide 104 on the metatarsal fixation platform 110 side of the cutting platform 2802.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the plurality of double cut guides 504 on the opposite side of the positioning slot 106 are angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), (θ₆), (θₓ₁), (θₓ₂), (θ_{y1}), (θ_{y2}), (θ_{z1}), and (θ_{z2}) created between the double cut guide 504 and the single cut guide 104 and the plurality of double cut guides 504, which may be any suitable angle. In various embodiments, the respective angles (θ₁), (θ₂), (θ₃), (θ₄), (θ₅), (θ₆), (θₓ₁), (θₓ₂), (θ_{y1}), (θ_{y1}), (θ_{z1}), and (θ_{z2}) may each be in the range of about 15° to about 75°.

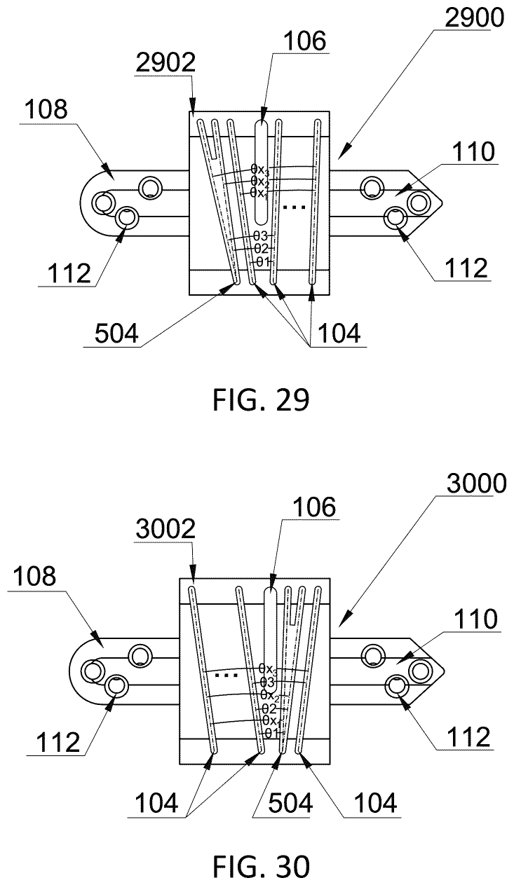

FIG. 29 is a diagram illustrating another embodiment of a surgical cutting block 2900 including a double cut guide 504 and a plurality of single cut guides 104. In this embodiment, the double cut guide 504 and one single cut guide 104 are located/positioned on a cutting platform 2902 toward the cuneiform fixation platform 108 and a plurality of single cut guides 104 are located/positioned on the cutting platform 2902 toward the metatarsal fixation platform 110. Further, the double cut guide 504 is located/positioned to the left of the single cut guide 104 on the cuneiform fixation platform 108 side of the cutting platform 2902.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction, the plurality of single cut guides 104 on the opposite side of the positioning slot 106 are angled in the direction as one another and a different direction than the double cut guide 504 and the single cut guide 104.

In additional or alternative embodiments, the double cut guide 504 and the single cut guide 104 are positioned on the cutting platform 1402 such that there is an angle (θ₁), (θ₂), (θ₃), (θₓ₁), (θₓ₂), and (θₓ₃) created between the double cut guide 504 and the single cut guide 104 and the other plurality of single cut guides 104, which may be any suitable angle. In various embodiments, the respective angles (θ₁), (θ₂), (θ₃), (θₓ₁), (θₓ₂), and (θₓ₃) may each be in the range of about 15° to about 75°.

FIG. 30 is a diagram illustrating another embodiment of a surgical cutting block 3000 including a double cut guide 504 and a plurality of single cut guides 104. In this embodiment, the double cut guide 504 and one single cut guide 104 are located/positioned on a cutting platform 3002 toward the metatarsal fixation platform 110 and a plurality of single cut guides 104 are located/positioned on the cutting platform 3002 toward the cuneiform fixation platform 108. Further, the double cut guide 504 is located/positioned to the left of the single cut guide 104 on the metatarsal fixation platform 110 side of the cutting platform 3002.

In some embodiments, the double cut guide 504 and the single cut guides 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction, the plurality of single cut guides 104 on the opposite side of the positioning slot 106 are angled in the direction as one another and a different direction than the double cut guide 504 and the single cut guide 104.

In additional or alternative embodiments, the double cut guide 504 and the single cut guide 104 are positioned on the cutting platform 1402 such that there is an angle (θ₁), (θ₂), (θ₃), (θₓ₁), (θₓ₂), and (θₓ₃) created between the double cut guide 504 and the single cut guide 104 and the other plurality of single cut guides 104, which may be any suitable angle. In various embodiments, the respective angles (θ₁), (θ₂), (θ₃), (θₓ₁), (θₓ₂), and (θₓ₃) may each be in the range of about 15° to about 75°.

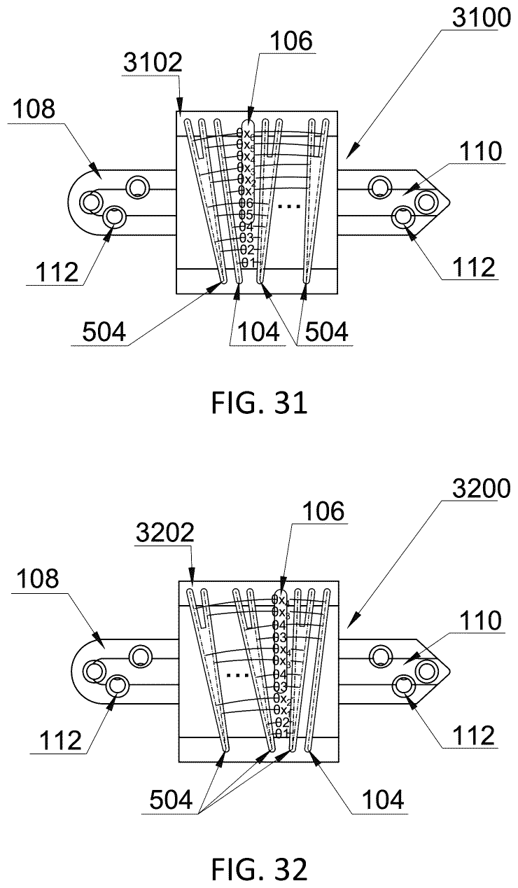

FIG. 31 is a diagram illustrating one embodiment of a surgical cutting block 3100 including a plurality of double cut guides 504 and a single cut guide 104. In this embodiment, one double cut guide 504 and the single cut guide 104 are located/positioned on a cutting platform 3102 toward the cuneiform fixation platform 108 and a plurality of double cut guides 504 are located/positioned on the cutting platform 3102 toward the metatarsal fixation platform 110. Further, the double cut guide 504 is located/positioned to the left of the single cut guide 104 on the cuneiform fixation platform 108 side of the cutting platform 3102.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the plurality of double cut guides 504 on the opposite side of the positioning slot 106 are angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), (θ₆), (θₓ₁), (θₓ₂), (θₓ₃), (θₓ₄), (θₓ₅), and (θₓ₆) created between the double cut guide 504 and the single cut guide 104 and the plurality of double cut guides 504, which may be any suitable angle. In various embodiments, the respective angles (θ₁), (θ₂), (θ₃), (θ₄), (θ₅), (θ₆), (θₓ₁), (θₓ₂), (θₓ₃), (θₓ₄), (θₓ₅), and (θₓ₆) may each be in the range of about 15° to about 75°.

FIG. 32 is a diagram illustrating another embodiment of a surgical cutting block 3200 including a plurality of double cut guides 504 and a single cut guide 104. In this embodiment, one double cut guide 504 and the single cut guide 104 are located/positioned on a cutting platform 3202 toward the metatarsal fixation platform 110 and a plurality of double cut guides 504 are located/positioned on the cutting platform 3202 toward the cuneiform fixation platform 108. Further, the double cut guide 504 is located/positioned to the left of the single cut guide 104 on the metatarsal fixation platform 110 side of the cutting platform 3202.

In some embodiments, the double cut guides 504 and the single cut guide 104 are angled in the same direction (e.g., angled toward the left or toward the right with respect to the user (e.g., a medical professional)). In the illustrated embodiment, the double cut guide 504 and the single cut guide 104 on the same side of the positioning slot 106 are angled in the same direction and the plurality of double cut guides 504 on the opposite side of the positioning slot 106 are angled in a different direction.

In additional or alternative embodiments, the double cut guide 504 and the single cut guide 104 are positioned on the cutting platform 1402 such that there is an angle (θ₁), (θ₂), (θ₃), (θ₄), (θ₅), (θ₆), (θₓ₁), (θₓ₂), (θₓ₃), (θₓ₄), (θₓ₅), and (θₓ₆) created between the double cut guide 504 and the single cut guide 104 and the plurality of double cut guides 504, which may be any suitable angle. In various embodiments, the respective angles (θ₁), (θ₂), (θ₃), (θ₄), (θ₅), (θ₆), (θₓ₁), (θₓ₂), (θₓ₃), (θₓ₄), (θₓ₅), and (θₓ₆) may each be in the range of about 15° to about 75°.

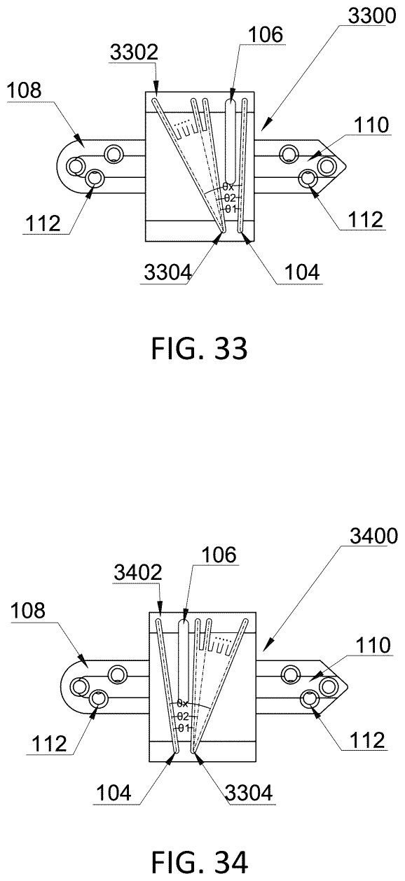

FIG. 33 is a diagram illustrating one embodiment of a surgical cutting block 3300 including a multi-cut guide 3304 and a single cut guide 104. In this embodiment, the multi-cut guide 3304 is located/positioned on the cutting platform 102 toward the cuneiform fixation platform 108 and the single cut guide 104 is located/positioned on a cutting platform 3202 toward the metatarsal fixation platform 110.

The multi-cut guide 3304 includes three or more paths that can include any suitable size and/or width capable of accepting a surgical cutting tool (e.g., a scalpel, surgical knife, etc.) and provide a respective path for the surgical cutting tool to follow while the surgical cutting tool is being used to produce an incision in a patient. The multi-cut guide 3304 includes an angle, which can be any suitable angle (e.g., an angle in the range of about one degree (1°) to about 90 degrees (90°)) with respect to any suitable reference point that can provide a predetermined and/or target path for the surgical cutting tool to follow while the surgical cutting tool is cutting a patient and/or performing a surgical procedure. Further, the angle between each respective pair of paths may be any suitable angle.

In some embodiments, the multi-cut guide 3304 and the single cut guide 104 are angled in the same direction (e.g., angled toward the left or toward the right with respect to the user (e.g., a medical professional)). In the illustrated embodiment, the multi-cut guide 3304 and the single cut guide 104 are angled in different directions.

In additional or alternative embodiments, the multi-cut guide 3304 and the single cut guide 104 are positioned on the cutting platform 1402 such that there is an angle (θ₁), (θ₂), and (θₓ) created between the multi-cut guide 3304 and the single cut guide 104, which may be any suitable angle. In various embodiments, the respective angles (θ₁), (θ₂), and (θₓ) may each be in the range of about 15° to about 75°.

FIG. 34 is a diagram illustrating another embodiment of a surgical cutting block 3400 including a multi-cut guide 3304 and a single cut guide 104. In this embodiment, the multi-cut guide 3304 is located/positioned on a cutting platform 3402 toward the metatarsal fixation platform 110 and the single cut guide 104 is located/positioned on the cutting platform 3402 toward the cuneiform fixation platform 108.

In some embodiments, the multi-cut guide 3304 and the single cut guide 104 are angled in the same direction (e.g., angled toward the left or toward the right with respect to the user (e.g., a medical professional)). In the illustrated embodiment, the multi-cut guide 3304 and the single cut guide 104 are angled in different directions.

In additional or alternative embodiments, the multi-cut guide 3304 and the single cut guide 104 are positioned on the cutting platform 1402 such that there is an angle (θ₁), (θ₂), and (θₓ) created between the multi-cut guide 3304 and the single cut guide 104, which may be any suitable angle. In various embodiments, the respective angles (θ₁), (θ₂), and (θₓ) may each be in the range of about 15° to about 75°.

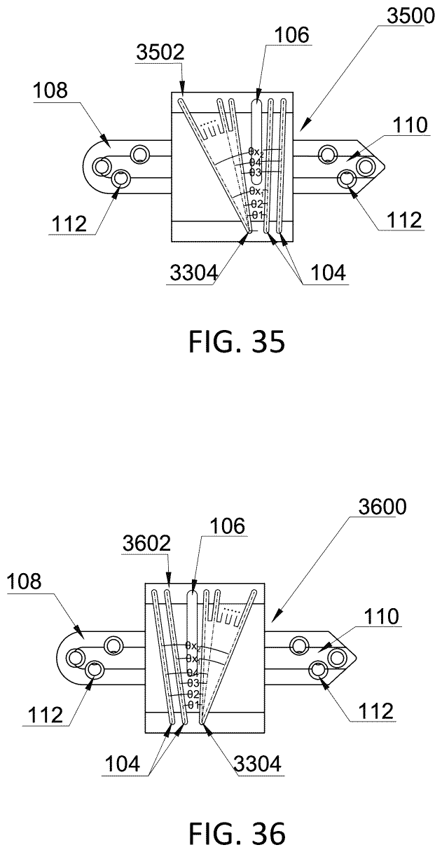

FIG. 35 is a diagram illustrating one embodiment of a surgical cutting block 3500 including a multi-cut guide 3304 and a plurality of single cut guides 104. In this embodiment, the multi-cut guide 3304 is located/positioned on a cutting platform 3502 toward the cuneiform fixation platform 108 and the plurality of single cut guides 104 are located/positioned on the cutting platform 3502 toward the metatarsal fixation platform 110.

In some embodiments, the multi-cut guide 3304 and the plurality of single cut guides 104 are angled in the same direction (e.g., angled toward the left or toward the right with respect to the user (e.g., a medical professional)). In the illustrated embodiment, the multi-cut guide 3304 and the plurality of single cut guides 104 are angled in different directions.

In additional or alternative embodiments, the multi-cut guide 3304 and the plurality of single cut guides 104 are positioned on the cutting platform 1402 such that there is an angle (θ₁), (θ₂), (θ₃), (θ₄), (θₓ₁), and (θₓ₂) created between the multi-cut guide 3304 and the plurality of single cut guides 104, which may be any suitable angle. In various embodiments, the respective angles (θ₁), (θ₂), (θ₃), (θ₄), (θₓ₁), and (θₓ₂) may each be in the range of about 15° to about 75°.

FIG. 36 is a diagram illustrating another embodiment of a surgical cutting block 3600 including a multi-cut guide 3304 and a plurality of single cut guides 104. In this embodiment, the multi-cut guide 3304 is located/positioned on a cutting platform 3602 toward the metatarsal fixation platform 110 and the plurality of single cut guides 104 are located/positioned on the cutting platform 3602 toward the cuneiform fixation platform 108.

In some embodiments, the multi-cut guide 3304 and the plurality of single cut guides 104 are angled in the same direction (e.g., angled toward the left or toward the right with respect to the user (e.g., a medical professional)). In the illustrated embodiment, the multi-cut guide 3304 and the plurality of single cut guides 104 are angled in different directions.

In additional or alternative embodiments, the multi-cut guide 3304 and the plurality of single cut guides 104 are positioned on the cutting platform 1402 such that there is an angle (θ₁), (θ₂), (θ₃), (θ₄), (θₓ₁), and (θₓ₂) created between the multi-cut guide 3304 and the plurality of single cut guides 104, which may be any suitable angle. In various embodiments, the respective angles (θ₁), (θ₂), (θ₃), (θ₄), (θₓ₁), and (θₓ₂) may each be in the range of about 15° to about 75°.

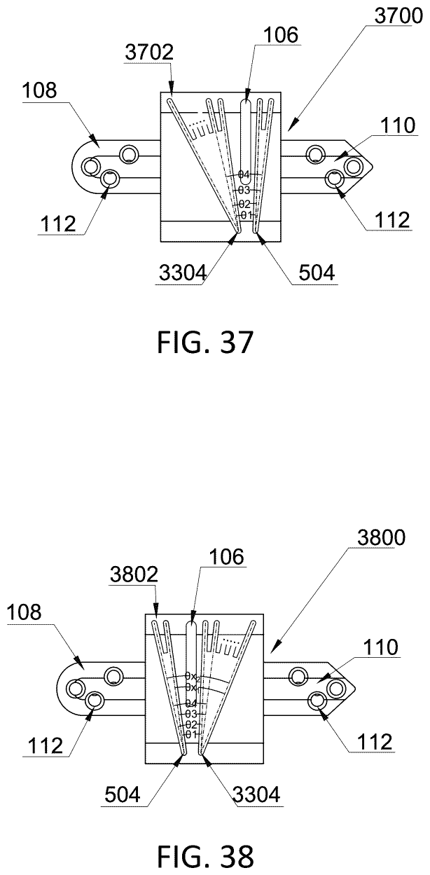

FIG. 37 is a diagram illustrating one embodiment of a surgical cutting block 3700 including a multi-cut guide 3304 and a double cut guide 504. In this embodiment, the multi-cut guide 3304 is located/positioned on a cutting platform 3702 toward the cuneiform fixation platform 108 and the double cut guide 504 is located/positioned on the cutting platform 3702 toward the metatarsal fixation platform 110.

In some embodiments, the multi-cut guide 3304 and the double cut guide 504 are angled in the same direction (e.g., angled toward the left or toward the right with respect to the user (e.g., a medical professional)). In the illustrated embodiment, the multi-cut guide 3304 and the double cut guide 504 are angled in different directions.

In additional or alternative embodiments, the multi-cut guide 3304 and the double cut guide 504 are positioned on the cutting platform 1402 such that there is an angle (θ₁), (θ₂), (θ₃), (θ₄), (θₓ₁), and (θₓ₂) created between the multi-cut guide 3304 and the double cut guide 504, which may be any suitable angle. In various embodiments, the respective angles (θ₁), (θ₂), (θ₃), (θ₄), (θₓ₁), and (θₓ₂) may each be in the range of about 15° to about 75°.

FIG. 38 is a diagram illustrating another embodiment of a surgical cutting block 3800 including a multi-cut guide 3304 and a double cut guide 504. In this embodiment, the multi-cut guide 3304 is located/positioned on a cutting platform 3802 toward the metatarsal fixation platform 110 and the double cut guide 504 is located/positioned on the cutting platform 3802 toward the cuneiform fixation platform 108.

In some embodiments, the multi-cut guide 3304 and the double cut guide 504 are angled in the same direction (e.g., angled toward the left or toward the right with respect to the user (e.g., a medical professional)). In the illustrated embodiment, the multi-cut guide 3304 and the double cut guide 504 are angled in different directions.

In additional or alternative embodiments, the multi-cut guide 3304 and the double cut guide 504 are positioned on the cutting platform 1402 such that there is an angle (θ₁), (θ₂), (θ₃), (θ₄), (θₓ₁), and (θₓ₂) created between the multi-cut guide 3304 and the double cut guide 504, which may be any suitable angle. In various embodiments, the respective angles (θ₁), (θ₂), (θ₃), (θ₄), (θₓ₁), and (θₓ₂) may each be in the range of about 15° to about 75°.

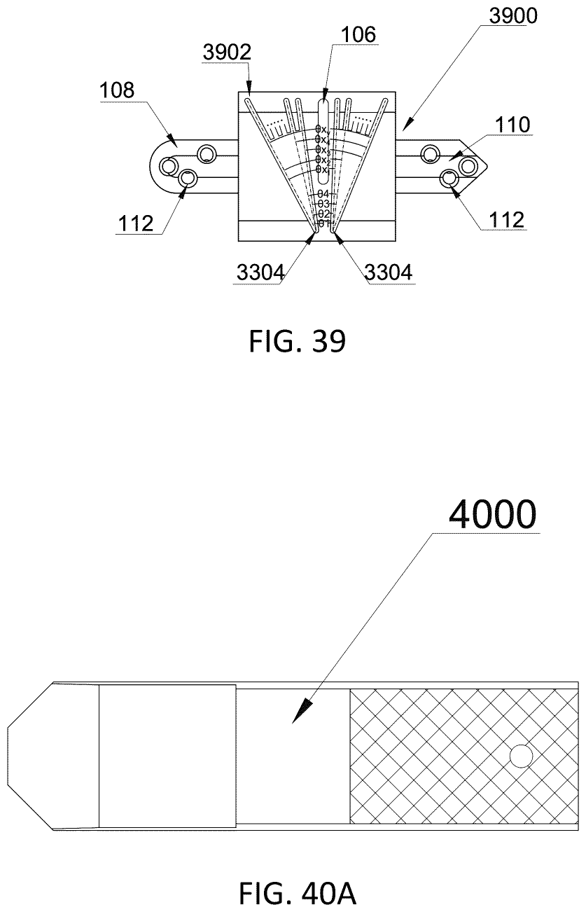

FIG. 39 is a diagram illustrating one embodiment of a surgical cutting block 3900 including a plurality of multi-cut guides 3304. In this embodiment, a multi-cut guide 3304 is located/positioned on a cutting platform 3902 on both sides of the positioning slot 106.

In some embodiments, the multi-cut guides 3304 are angled in the same direction (e.g., angled toward the left or toward the right with respect to the user (e.g., a medical professional)). In the illustrated embodiment, the multi-cut guides 3304 are angled in different directions.

In additional or alternative embodiments, the multi-cut guides 3304 are positioned on the cutting platform 1402 such that there is an angle (θ₁), (θ₂), (θ₃), (θ₄), (θₓ₁), (θₓ₂), (θₓ₃), (θₓ₄), and (θₓ₅) created between the multi-cut guides 3304, which may be any suitable angle. In various embodiments, the respective angles (θ₁), (θ₂), (θ₃), (θ₄), (θₓ₁), (θₓ₂), (θₓ₃), (θₓ₄), and (θₓ₅) may each be in the range of about 15° to about 75°.

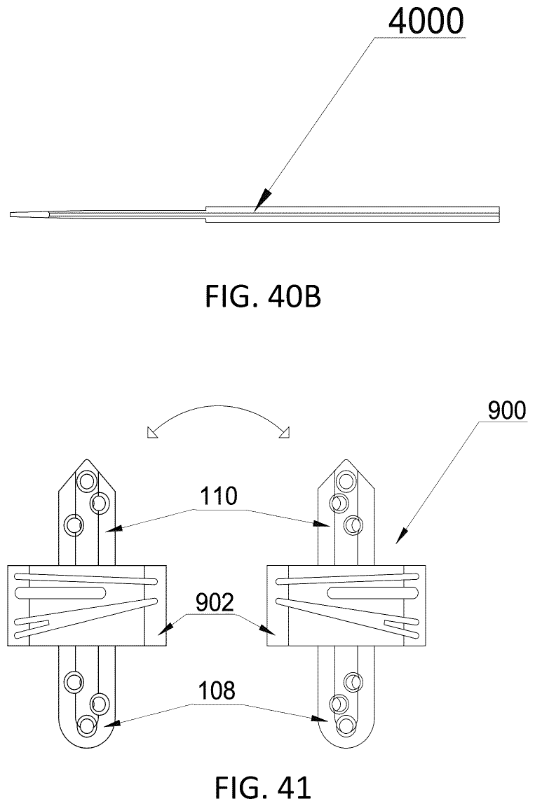

FIG. 40A is a diagram illustrating one embodiment of a positioning fin 4000. The positioning guide 4000 may include any shape and/or size that can be accommodated by a positioning slot 106.

Further, the positioning guide 4000 may include and/or be formed of any suitable material. In some embodiments, the positioning guide 4000 includes a sterilizable material.

FIG. 40B is a diagram illustrating a profile view of the positioning fin illustrated in FIG. 40A. FIG. 41 is a diagram illustrating that opposite sides of surgical cutting blocks 100 through 3900 can be used on the left foot and/or the right foot of a patient.

The positioning guide 4000 and the positioning slot 106 are configured to cooperatively prepare a cuneiform-metatarsal joint for a surgical procedure. In some embodiments, the positioning guide 4000 and the positioning slot 106 are configured to cooperatively guide the positioning of a surgical cutting block (e.g., surgical cutting blocks 100 through 3900) at a cuneiform-metatarsal joint in preparation for a surgical procedure (e.g., a surgery to correct a bunion).

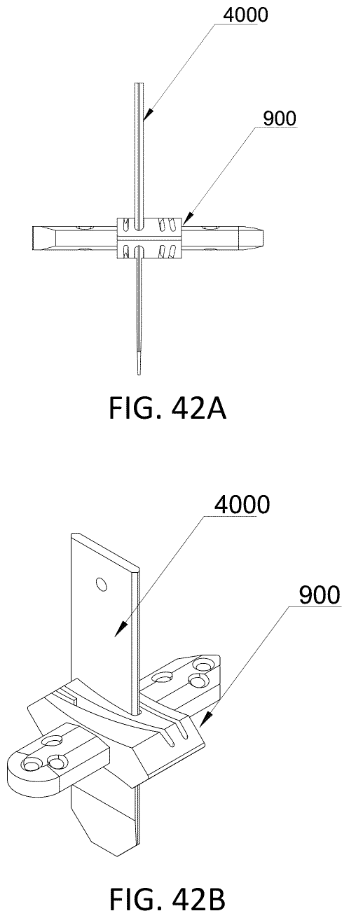

A cutting platform (e.g., cutting platforms 102 through 3902) allows a cut guide (e.g., single cut guide(s) 104, double cut guide(s) 504, and/or multi-cut guide(s) 3304) to sit close to a bone surface. Surgical cutting blocks 100 through 3900 may be placed in the correct position by placing the positioning fin 4000 through the positioning slot 106 in the cutting portion (see, e.g., FIGS. 42A & 42B). The positioning fin 4000 is placed through the positioning slot 106 to allow the cut guide to placed centrally on a joint.

The cut guides are held in place using pins driven through the wire holes 112 into the cuneiform and metatarsal. Some wire holes 112 may include an angle while others are perpendicular to the surface of a cut guide.

The embodiments may be practiced in other specific forms. The described embodiments are to be considered in all respects only as illustrative and not restrictive. The scope of the technology is, therefore, indicated by the appended claims rather than by the foregoing description. All changes which come within the meaning and range of equivalency of the claims are to be embraced within their scope.

1. A surgical cutting block, comprising:
a cutting platform including a positioning aperture formed on the cutting platform, the positioning platform dividing the cutting platform into a first side and a second side; and
a first plurality of single cut guides formed on the first side.

2. The surgical cutting block of claim 1, further comprising a single cut guide formed on the second side.

3. The surgical cutting block of claim 1, further comprising a second plurality of single cut guides formed on the second side.

4. The surgical cutting block of claim 3, wherein at least one of the first plurality of single cut guides and the second plurality of single cut guides comprises three or more single cut guides.

5. A surgical cutting block, comprising:
a cutting platform including a positioning aperture formed on the cutting platform, the positioning platform dividing the cutting platform into a first side and a second side; and
a first multi-cut guide formed on the first side.

6. The surgical cutting block of claim 5, further comprising a single cut guide formed on the second side.

7. The surgical cutting block of 5, further comprising a double cut guide formed on the second side.

8. The surgical cutting block of claim 5, further comprising a second multi-cut guide formed on the second side.

9. The surgical cutting block of claim 8, wherein at least one of the first multi-cut guide and the second multi-cut guide includes three or more cut guides.

10. The surgical cutting block of claim 5, wherein the first multi-cut guide includes three or more cut guides.
